(19) United States
(12) Patent Application Publication
(10) Pub. No.: US 2002/0150895 A1
(43) Pub. Date: Oct. 17, 2002

(54) Method and apparatus for filtering and extending RNA alignment coverage

(76) Inventor: Raymond Wheeler, Berkeley, CA (US)

Correspondence Address: GLENN PATENT GROUP, 3475 EDISON WAY, SUITE L, MENLO PARK, CA, 94025, US

(21) Appl. No.: 09/747,440

(22) Filed: Dec. 22, 2000

(51) Int. Cl. C12Q 1/68; G06F 19/00

(52) U.S. Cl. 435/6; 702/19

(57) ABSTRACT

A technique is provided that is capable of extending a typically sparse RNA alignment coverage, without creating redundant or improbable alignments. At a high level, the invention provides a two-step process. The first step is to combine and catenate all combinations of overlapping alignments that agree with each other. The second step is to extend the boundaries of overlapping alignments that agree with their first and last exons.

### BACKGROUND OF THE INVENTION

1. Technical Field

The invention relates to the analysis of combination of RNA derived sequence fragments, hereinafter referred to as RNA. More particularly, the invention relates to a method and apparatus for filtering and extending RNA alignment coverage.

2. Description of the Prior Art

Expressed sequence tags (EST) and mRNA sequences can be aligned to genomic for two purposes. One purpose is to enhance gene prediction, and the other is to examine alternative splice forms of a gene. In the case of enhancing gene prediction, a set of non-overlapping alignments can be fed to a gene prediction algorithm, which uses them to give higher weight to the probability of there being a gene in the covered areas. With alternative gene splice forms, each alignment variant is fed to the gene predictor one at a time, which then gives greater weight to the input splice form.

These two purposes can be at odds with each other. In the case of enhancing gene prediction, it is desirable to extend the alignment coverage as much as possible to improve the gene prediction. On the other hand, each run of the gene prediction algorithm is CPU intensive. It is therefore preferable to run the algorithm over the same genomic region only a minimum number of times. In the case of alternative splice forms, running the algorithm repeatedly over the same genomic area is unavoidable because it can only take non-overlapping alignments as input. However, a problem arises when the different splice forms are extended and combined in such a way that a combinatorial explosion results. For example, if a gene has four areas in which there exist four splice variants in each area, a naive combination of these would result in 256 alternative splice forms. While this may be conceivable, it does not seem likely. See FIG. 1, in which rectangles represent matching blocks, i.e. exons, while lines represent gaps in the alignment, i.e. introns. FIG. 1 shows four aligned RNAs (A, B, C, D) from a gene with four areas containing four splice variations each. Permitting all possible exon combinations would yield 256 possibilities. These issues give rise to the need for an approach that is capable of extending a typically sparse RNA alignment coverage, without creating redundant or improbable alignments.

### SUMMARY OF THE INVENTION

The invention provides a technique that is capable of extending a typically sparse RNA alignment coverage, without creating redundant or improbable alignments. At a high level, the invention provides a two-step process. The first step is to combine and catenate all combinations of overlapping alignments that agree with each other. The second step is to extend the boundaries of overlapping alignments that agree with their first and last exons.

### BRIEF DESCRIPTION OF THE DRAWINGS

FIG. 1 shows four aligned RNAs from a gene with four areas containing four splice variations each;

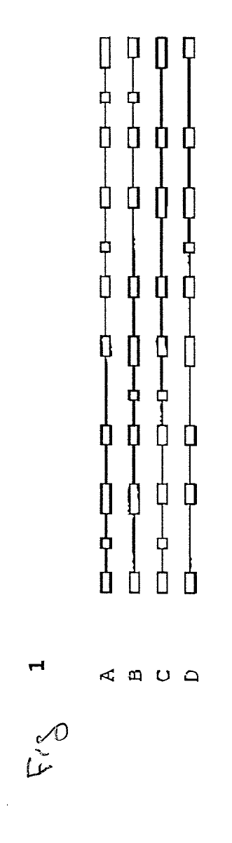

FIG. 2 is a flow diagram showing a technique for filtering and extending RNA alignment coverage according to the invention;

FIG. 3 shows a sample run, in which rectangles represent matching blocks, i.e. exons, while the lines represent gaps in the alignment, i.e. introns;

FIG. 4 shows splice variants that would be suitable for combining with other well-supported alternative splicings;

FIG. 5 shows a hypothetical run of a liberal alternative splicing algorithm that creates four possible splice variants where only one is expected;

FIG. 6 shows a hypothetical run of a liberal alternative splicing algorithm that creates unsupported exon combinations;

FIG. 7 is a flow diagram showing a preprocessing and filtering technique according to the invention;

FIG. 8 shows a sample input;

FIG. 9 is a flow diagram showing a merging technique according to the invention;

FIG. 10 shows different outputs, depending on when redundant alignment B is discarded;

FIG. 11 is a flow diagram showing an extending technique according to the invention;

FIG. 12 shows a sample run of an extending step; and

FIG. 13 is an example showing the invention used to filter out redundant alignments.

### DETAILED DESCRIPTION OF THE INVENTION

Splice sites are pairs of nucleotides that indicate the location of an intron/exon cleavage site. The 5′ site is the upstream, donor site, while the 3′ site is the downstream, acceptor site. Together, 5′/3′ splice sites delineate the boundaries between exons and introns. Two alignments conflict when they disagree about the location of one or more 5′/3′ splice sites in a given genomic region. Two alignments agree when they overlap and there is no disagreement about the location of their 5′/3′ splice sites. Two alignments overlap if they are aligned to overlapping sections of the genomic.

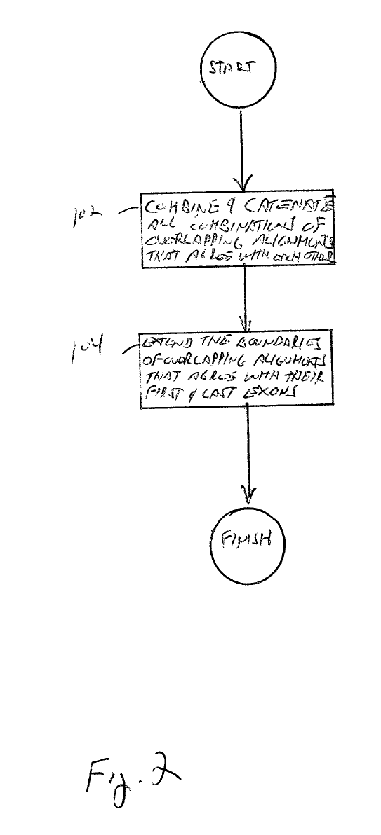

FIG. 2 is a flow diagram showing a technique for filtering and extending RNA alignment coverage according to the invention. The invention herein is applicable to any combination of RNA derived sequence fragments, for example (but not for purposes of limiting the applicability of the invention to other sequences and sequence fragments) RNA derived sequence fragments comprising any of EST's, partial cDNA's and full-length cDNA's.

At a high level, the invention provides a two-step process.

The first step (102) is to combine and catenate all combinations of overlapping alignments that agree with each other.

The second step (104) is to extend the boundaries of overlapping alignments that agree with their first and last exons.

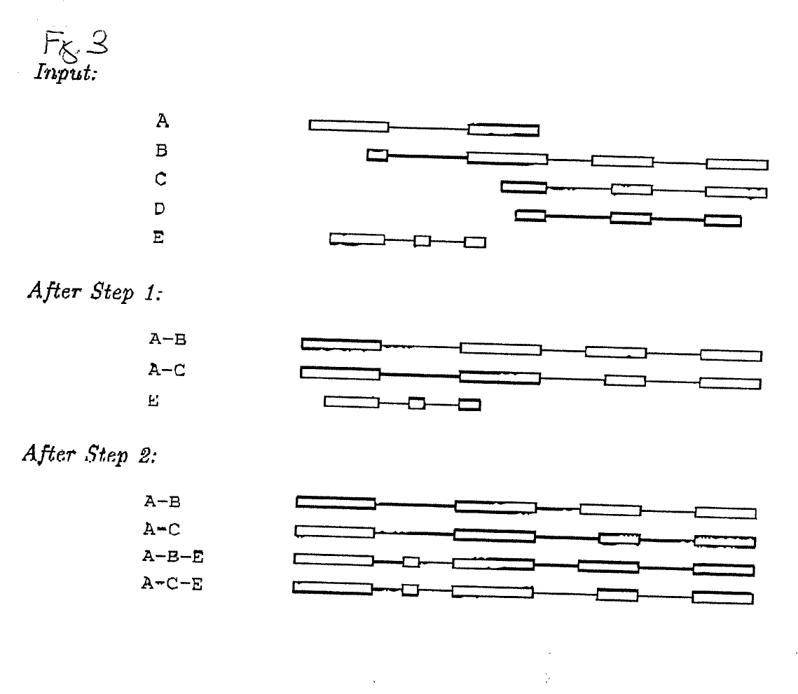

FIG. 3 shows a sample run, in which rectangles represent matching blocks, i.e. exons, while the lines represent gaps in the alignment, i.e. introns.

In FIG. 3, step 1 (Input), the alignment E does not merge with any other alignments. This is because its second exon does not agree with any other alignment.

Step 1 serves the purpose of filtering out many redundant alignments, as well as combining and expanding alignments that support a self-consistent splicing. There are occasions however, when the RNA evidence for an alternative splicing is local to a particular part of the gene, and is not able to merge with any other alignments due to its irregular set of splice sites (see FIG. 3, alignment E). It would be useful to have this kind of alternative splicing presented in the context of the entire gene, and its other splice variants (as in FIG. 3, alignments A-B-E and A-C-E). However, it is important to be careful not to create any exotic combinations that are not well-supported by the alignment evidence.

Assessing Splice Variant Combinations

There are many other splice variants such as shifted slice sites, and variable exons that it is desirable to see combined with the other variations (see Schmucker et al., Drosophila dscam is an axon guidance receptor exhibiting extraordinary molecular diversity, Cell 101, pp. 671-684 (2000)).

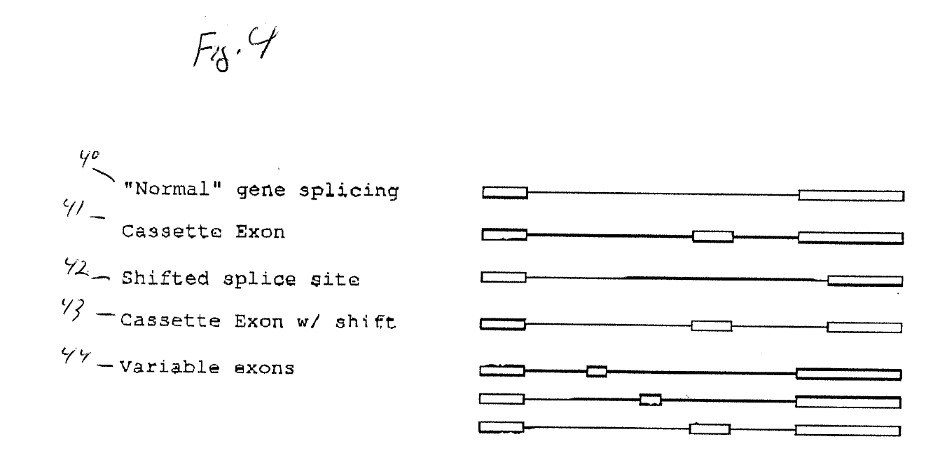

FIG. 4 shows splice variants that would be suitable for combining with other well-supported alternative splicings in accordance with the invention disclosed herein. Thus, FIG. 4 shows a normal gene splicing 40, a cassette exon 41, a shifted splice site 42, a cassette exon with a shift 43, and variable exons 44. In FIG. 4, the term ‘Cassette exon with shift’ is explicitly listed because the inclusion of a cassette exon often causes a frame shift, leading to the presence of one or more nearby shifted splice sites.

There are other splice variants however, that if combined, would lead to debatable alternative splicings. For example, consider an alignment that included an RNA that was only partially spliced. It would be possible to combine spliced RNA with it in such a way that would create many undesirable splicings.

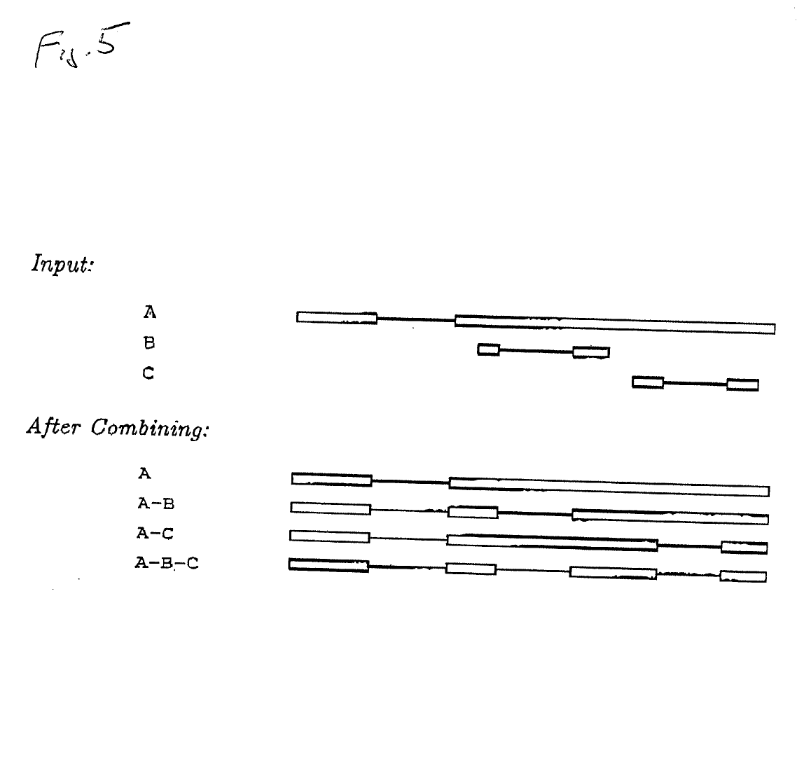

FIG. 5 shows a hypothetical run of a liberal alternative splicing algorithm that creates four possible splice variants where only one is expected. In FIG. 5, there is probably only one gene splicing with all three introns. By being very liberal in accepting alternative splice forms, four variants can be inferred, i.e. the last variant (A-B-C), which is expected, plus the first three dubious variants (A, A-B, and A-C).

Note that if the alignment A spanned three introns instead of two, then there would be seven dubious exon combinations. This situation has the possibility for an exponential explosion in possible splice combinations.

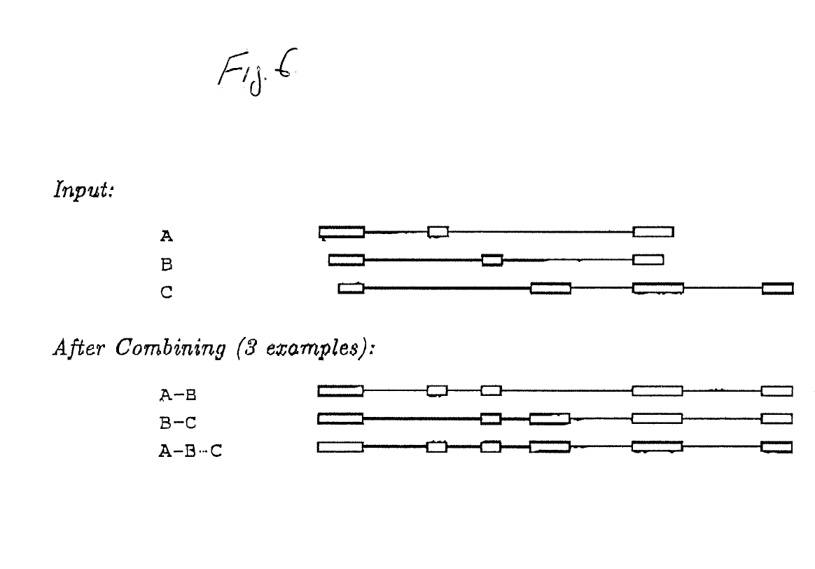

With regard to the example of variable exons shown in FIG. 4, consider that is given the three alignments shown as input, it is not desirable to create unsupported exon combinations (see FIG. 6). As before, this situation has the potential for a combinational explosion in splice possibilities, most of which would be suspect.

The solution to this problem in the herein disclosed algorithm is to preserve the exon configurations that are found in the input. Rather than adopt the notion of combining different splice variations, the idea is to extend the given variations. Thus, each different, shorter variation is extended in a manner identical to alignments A-B and A-C. This is because alignment E agrees with the others in their first and last overlapping exons. This procedure is discussed in greater detail below.

Algorithm

FIG. 6 shows a hypothetical run of a liberal alternative splicing algorithm that creates unsupported exon combinations. The output would actually have seven alternative splicings, i.e. three splicings reflecting the input, and four fabricated splicings.

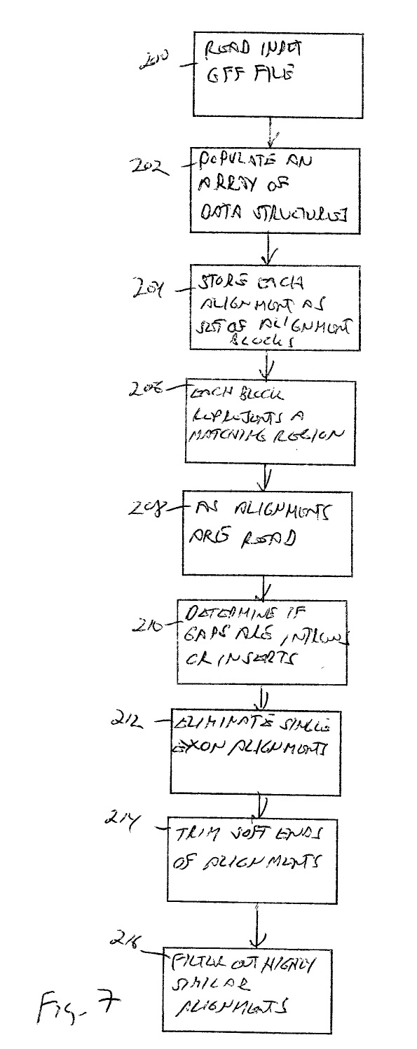

FIG. 7 is a flow diagram showing a preprocessing and filtering technique according to the invention. The presently preferred embodiment of the invention comprises an algorithm that begins by reading a file containing alignments of exon annotations to genomic sequence (200) and populating an array of data structures (202), one for each alignment. Each alignment is stored as a set of alignment blocks (204), each block representing a matching S region between the given RNA and the genomic (206). The blocks are considered exons, and the gaps between them are considered introns. For purposes of the discussion herein, the 5′/3′ splice sites are referred to as hard edges, and the two ends of the alignment are referred to as soft ends.

As the alignments are read (208), some data filtering and pre-processing is performed.

1. Determine if gaps are introns or inserts (210).

2. Eliminate single exon alignments (212).

3. Trim soft ends of alignments (214).

4. Safely filter out highly similar alignments (216).

First, gaps in the alignment blocks that are less than or equal to twenty nucleotides are considered inserts instead of introns, and the two adjacent blocks are subsequently combined into one. This number is a highly conservative, and somewhat arbitrary, educated guess at the length of the smallest human intron. Those skilled in the art will appreciate that other values may be chosen in connection with practice of the invention. Second, once an entire alignment has been read and all inserts removed, the alignment is discarded if it consists of only a single exon.

Third, the soft ends of the alignment are trimmed by ten nucleotides. This is a heuristic for correcting a possible alignment error, in which a small number of terminal nucleotides are aligned to a lucky adjacent intron, instead of the distant correct exon. Those skilled in the art will appreciate that other values may be chosen in connection with practice of the invention.

Lastly, once all of the multi-exon alignments have been read and trimmed, there is a filtering step to cut down on running time.

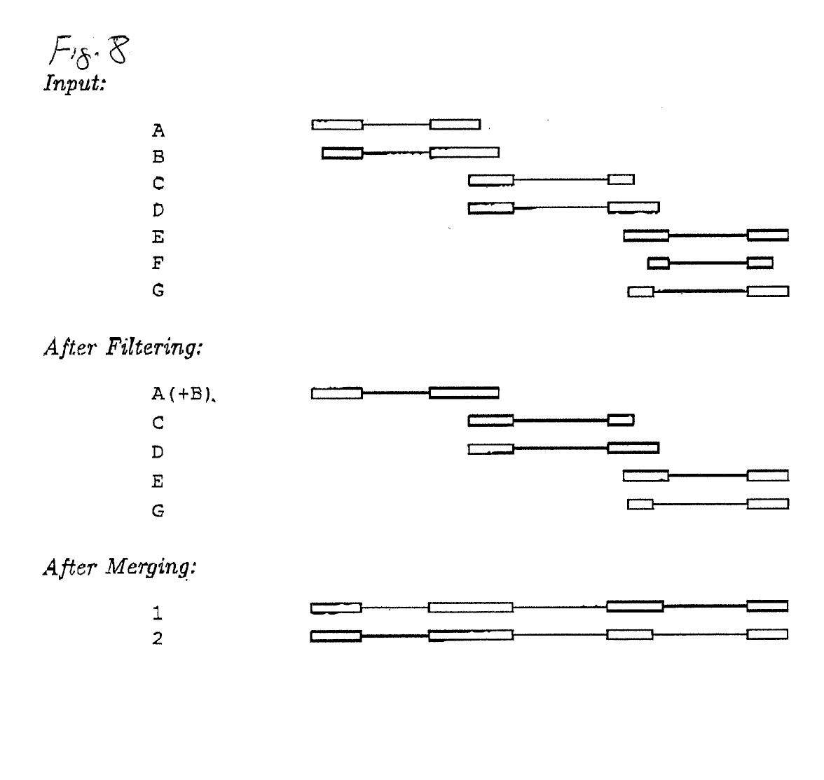

FIG. 8 shows a sample input. Multiple alignments that are very similar can lead to an exponential number of possible mergings. Filtering out similar alignments can be tricky. Consider FIG. 8. There are three pairs of very similar alignments. In this example, alignments A and B can each merge with alignments C and D. The resulting alignments A-C, A-D, B-C, and B-D, can be merged with alignments E, F, and G to create eight combinations in a situation where only two combinations are necessary. A simple solution is to throw out similar alignments. Two alignments are similar if they are on the same strand, and have similar number of agreeing exons. This however, could have undesirable side-effects.

In FIG. 8, one could eliminate shorter alignments A, C, and F because they are similar to alignments B, D, and E, respectively. This would leave a situation in which nothing could merge with alignment G, i.e. only A-C and B-C can align with G. On the other hand, one could eliminate alignment B, D, and E, but then nothing could merge with alignment F.

The solution is to remove similar alignments only if their soft ends are not on opposing sides of some other alignment's hard edge. In FIG. 8, neither alignment C nor D would be eliminated without altering any subsequent merging. To keep the alignments as long as possible, whenever a similar alignment is eliminated, the alignment that remains is stretched to the widest possible soft end points of the two alignments. The result is that for a set of many similar alignments whose soft ends are not near any hard edges, only one alignment remains after this filtering step. Furthermore, the soft ends of the remaining alignment are the widest possible among all of the similar alignments. The last part of FIG. 8 shows the results of filtering the current example. After filtering, there are only three merging combinations (A-C-E, A-D-E, and A-C-G), instead of eight.

Step 1-Merging

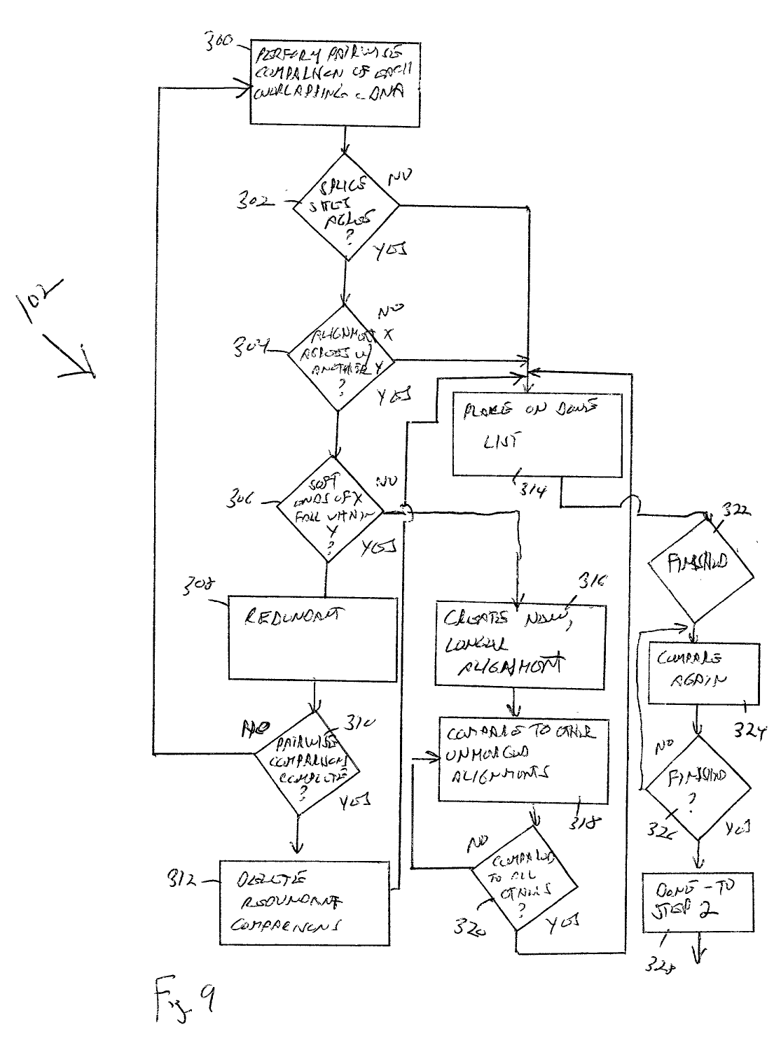

FIG. 9 is a flow diagram showing a merging technique according to the invention. The purpose of this step is to create larger alignments, while throwing out redundant alignments. To this end, the merge step performs a pairwise comparison of each overlapping RNA on the same strand (300). If their splice sites agree (302), then either a new, longer alignment is created out of the two (316; see FIG. 3 sequences A and B), or one of the alignments is labeled as redundant to the other (308), i.e. it is scheduled for deletion (312; see FIG. 3, sequences C and D).

An alignment X is redundant if it agrees with another alignment Y (304), and the soft ends of X fall completely within the soft ends of Y, inclusive (306). Redundant alignments are not deleted until after the pairwise comparisons are complete (310).

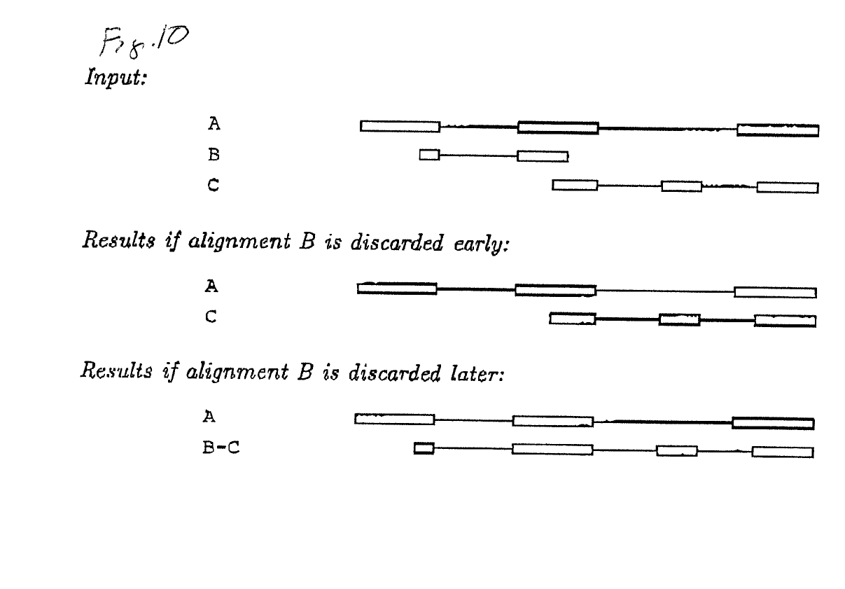

FIG. 10 shows different outputs, depending on when redundant alignment B is discarded. In FIG. 10, note that a smaller alignment can be redundant with respect to another larger alignment and still be able to merge with other alignments that the larger alignment cannot. Redundant pieces are still available for the pairwise comparisons, even though they are redundant with other alignments. This is why performing all pairwise comparisons is important. The significance of noting redundant alignments is that they should eventually be discarded because they are redundant.

Notice in FIG. 10 that if alignment B were completely ignored because it is redundant with alignment A, then it would not be available to merge with C.

Each newly created, merged alignment is also checked against the other, unmerged alignments in the same way (318): either merging once again, becoming labeled as redundant, or causing other alignments to be labeled redundant. Whenever an alignment, merged or not, has been compared to all other alignments (320), and is neither redundant nor merged it is placed on a list of Done alignments (314). Once all comparisons have been finished (322), the Done alignments are again compared pairwise to check for redundancies (324, 326, 328).

The possibility of redundant alignments within the Done list is seen in FIG. 4. There, alignment A merges with C, and the combination can not merge any further, so it is done. Alignment A also merges with D, and that combination is also done. Now both the A-C and A-D combination are on the Done list, yet the A-D combination is redundant.

In Table 1 below, the algorithm is guaranteed to terminate because the items on the todo list always begin their comparisons in the cdnaArray after the index where the merging took place. Therefore, attempting to compare the same merged alignment ad infinitum is impossible.

The merge portion is complete when the Done list has been cleared of redundant, merged alignments.

TABLE 1
Pseudo-code for merging algorithm

| / | * cdnaAray = array of cDNA alignments stored by increasing | | |
|---|---|---|---|
| | * | start points | |
| | * todo list = a list of merged alignments that are | | |
| | * | scheduled to be compared to the other | * |

alignments in cdnaArray. Associated with each

| | |
|---|---|
| * | merged alignment is an integer |
| * | array index indicating where in cdnaArray the |
| * | comparisons should begin. |

*/

bool merged;
for l = 0 to sizeOf (cdnaArray) {

for j = (l + 1) to sizeOf (cdnaArray) {

merged = 0;

if ( overlap (cdnaArray[l], cdnaArray[j])) {

merged l = merge (cdnaArray[l], cdnaArray[j], todo_list);

```
  }
}
if (!merged) {
  push(Done_list, cdnaArray[l]);
}
else {/* there must be a mered alignment on the todo_list */
  while (todo_list !=NULL) {
```

todo = pop(todo_list);
merged = 0;
for k = todo,dtart to size Of (cdnaArray) {
if (overlap (todo, cdnaArray [k])) {

merged l=merge (todo, cdnaArray [k], todo_list);

}
}
if (!merged) {
push(Done-list, todo);

}

}

}

}

Step 2—Extending

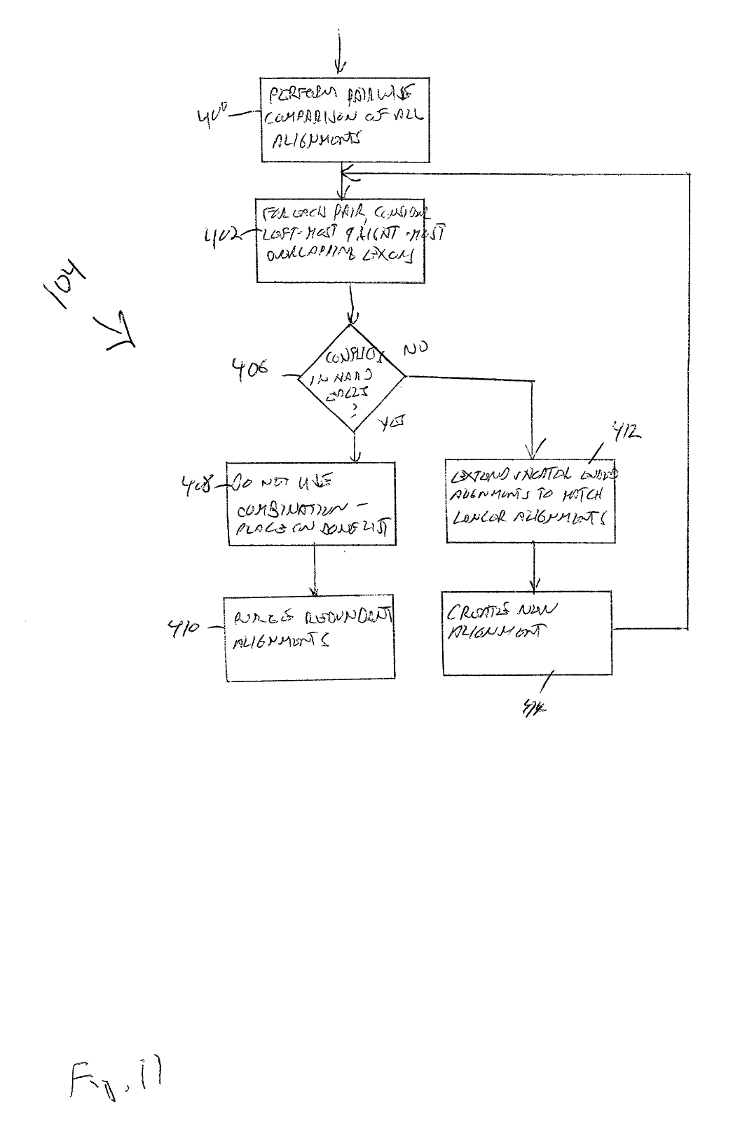

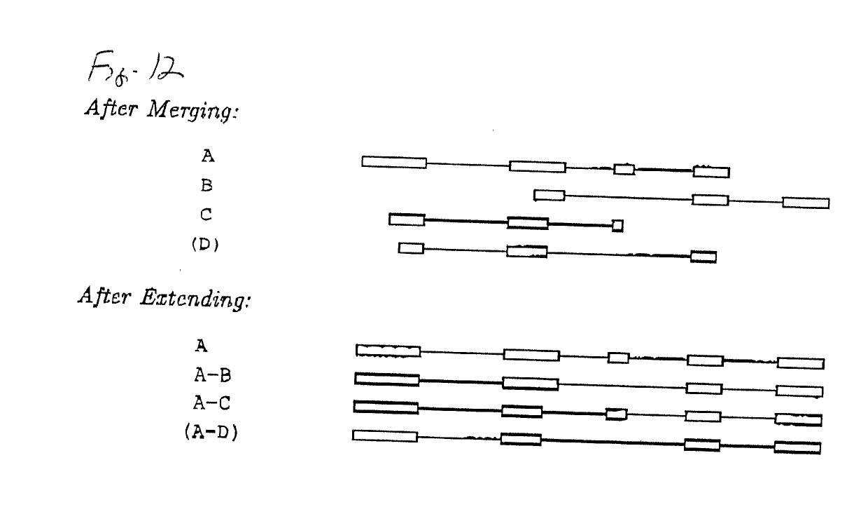

FIG. 11 is a flow diagram showing an extending technique according to the invention. FIG. 12 is a sample run of Step 2, extending. Combination (A-D) is possible only with the presence of alignment (D). It cannot be inferred from the other alignments.

This step also involves a pairwise comparison of all the remaining alignments (400). For each pair, the left-most and right-most overlapping exons are considered (402). If there are no conflicts on any of their hard edges (406), then the short ended alignments are extended in such a way that they match the longer alignments (412).

Notice in FIG. 12, after step 2, there would be no alignment that represents the exon combination of alignments B and C. This is because alignment C's final exon conflicts with B, and therefore the algorithm does not use this combination. If there were also an alignment D (shown in parentheses in FIG. 12) that was similar to C, only its final exon agreed with alignment B, then all four exon combinations would be considered.

The motivation for considering all pairs of alignments is that once an alignment has been extended, it has new opportunities to extend, and be extended by, other alignments. If an alignment can be extended, then a new alignment is created (414), and it is further compared against all overlapping alignments. If an alignment cannot be extended, it gets placed on a Done list (408), as before. After all comparisons are complete, the Done list is again purged of redundant alignments (410). The pseudo-code for this step is almost identical to the pseudo-code for the merging step, and is therefore omitted.

What remains after Step 2 is a minimal set of splice variant alignments, extended as far as they can given the alignment data.

The presently preferred embodiment of the invention thus comprises a conservative, yet effective technique that addresses the issue of combining and expanding aligned RNA's to create a concise set of inputs for a gene prediction algorithm. The preferred embodiment of the invention is capable of extending small RNA alignments that represent an alternative splicing into much larger alignments, consistent with the other well-supported splicings. This is done in a manner that does not create new exon combinations that are not supported by the given data. The preferred embodiment of the invention also outputs a set of alignments, each of which represents a different set of splice site combinations.

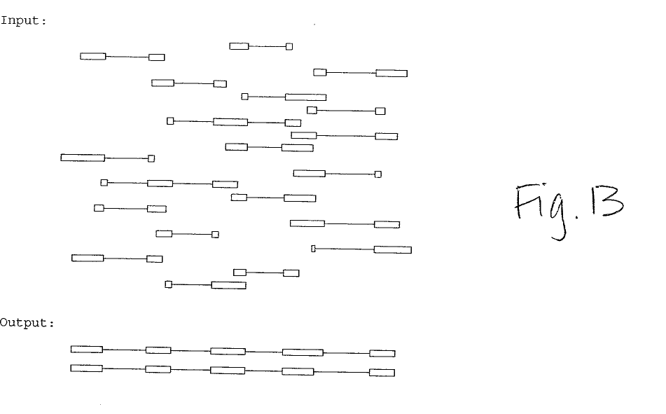

Although the invention is described herein with reference to the preferred embodiment, one skilled in the art will readily appreciate that other applications may be substituted for those set forth herein without departing from the spirit and scope of the present invention. In addition to the algorithm's ability to derive alternate splice form combinations, it has the capacity to filter out redundant alignments. As more and more RNA fragments are sequenced and recorded in genomic databases, the need for automated filtering of this data becomes greater and greater. Thus, in a situation where hundreds of RNA alignments overlap within the same genomic region, the algorithm could be used to cull out the possibly few useful splice forms, while purging the redundant alignment data (see FIG. 13). Accordingly, the invention should only be limited by the claims included below.

[image]

[image]

[image]

[image]

[image]

[image]

[image]

[image]

[image]

[image]

[image]

[image]

[image]

[image]

1. A method for extending sparse alignment coverage for any combination of RNA derived sequence fragments, comprising the steps of:
combining and catenating all combinations of overlapping alignments that agree with each other; and
extending boundaries of overlapping alignments that agree with their first and last exons.

2. The method of claim 1, further comprising the step of:
preprocessing and filtering.

3. The method of claim 2, wherein said preprocessing and filtering step comprises the steps of:
reading a file containing alignments of exon annotations to genomic sequence; and
populating an array of data structures, one for each alignment, wherein each alignment is stored as a set of alignment blocks, each block representing a matching region between a given RNA and the genomic.

4. The method of claim 3, wherein said blocks are considered exons, and gaps between them are considered introns.

5. The method of claim 4, wherein 5′/3′ splice sites are referred to as hard edges, and the two ends of an alignment are referred to as soft ends.

6. The method of claim 3, said preprocessing and filtering step further comprising the steps of:
determining if gaps are introns or inserts;
eliminating single exon alignments;
trimming soft ends of alignments; and
filtering out highly similar alignments.

7. The method of claim 5, wherein gaps in alignment blocks that are less than or equal to twenty nucleotides are considered inserts instead of introns, and wherein two adjacent blocks are subsequently combined into one.

8. The method of claim 7, wherein once an entire alignment has been read and all inserts removed, an alignment is discarded if it consists of only a single exon.

9. The method of claim 8, wherein soft ends of an alignment are trimmed by ten nucleotides.

10. The method of claim 9, wherein once all multi-exon alignments have been read and trimmed, there is a filtering step to cut down on running time.

11. The method of claim 1, further comprising the step of:
discarding similar alignments, wherein two alignments are similar if they are on a same strand and have similar number of agreeing exons.

12. The method of claim 1, further comprising the step of:
eliminating shorter alignments.

13. The method of claim 5, further comprising the step of:
removing similar alignments only if their soft ends are not on opposing sides of some other alignment's hard edge.

14. The method of claim 11, further comprising the step of:
whenever a similar alignment is eliminated, an alignment that remains is stretched to widest possible soft end points of said two alignments to keep said alignments as long as possible, wherein for a set of many similar alignments whose soft ends are not near any hard edges, only one alignment remains after said filtering step; and wherein soft ends of a remaining alignment are widest possible among all of similar alignments.

15. The method of claim 1, wherein said RNA derived sequence fragments comprise any of EST's, partial cDNA's and full-length cDNA's.

16. A method for extending sparse alignment coverage for any combination of RNA derived sequence fragments, comprising the steps of:
merging all combinations of overlapping alignments that agree with each other; and
extending boundaries of overlapping alignments that agree with their first and last exons.

17. The method of claim 16, wherein said merge step performs a pairwise comparison of each overlapping RNA sequence on a same strand;
wherein if their splice sites agree, then either a new, longer alignment is created out of said two sequences, or one of said alignments is labeled as redundant to the other and it is scheduled for deletion.

18. The method of claim 17, wherein an alignment X is redundant if it agrees with another alignment Y, and soft ends of X fall completely within soft ends of Y, inclusive.

19. The method of claim 18, wherein redundant alignments are not deleted until after said pairwise comparisons are complete.

20. The method of claim 19, wherein a smaller alignment can be redundant with respect to another larger alignment and still be able to merge with other alignments that a larger alignment cannot; and wherein redundant pieces are still available for said pairwise comparisons, even though they are redundant with other alignments.

21. The method of claim 20, wherein each newly created, merged alignment is also checked against other, unmerged alignments in a same way, either merging once again, becoming labeled as redundant, or causing other alignments to be labeled redundant.

22. The method of claim 21, wherein whenever an alignment, merged or not, has been compared to all other alignments, and is neither redundant nor merged it is placed on a list of Done alignments.

23. The method of claim 22, wherein once all comparisons have been finished, said Done alignments are again compared pairwise to check for redundancies.

24. The method of claim 16, wherein said merging step comprises the following algorithm:

| / | * cdnaAray = array of cDNA alignments stored by increasing | | |
|---|---|---|---|
| | * | start points | |
| | * todo list = a list of merged alignments that are | | |
| | * | scheduled to be compared to the other | * |

alignments in cdnaArray. Associated with each

| | |
|---|---|
| * | merged alignment is an integer |
| * | array index indicating where in cdnaArray the |
| * | comparisons should begin. |

*/
bool merged;
for l = 0 to sizeOf (cdnaArray) {
for j = (l + 1) to sizeOf (cdnaArray) {
merged = 0;
if ( overlap (cdnaArray[l], cdnaArray[j])) {
merged l = merge (cdnaArray[l], cdnaArray[j], todo_list);
```
  }
}
if (!merged) {
  push(Done_list cdnaArray[l]));
}
else {/* there must be a mered alignment on the todo_{—list */}
  while (todo_list !=NULL) {
```
todo = pop(todo_list);
merged = 0;
for k = todo,dtart to size Of (cdnaArray) {
if (overlap (todo, cdnaArray [k])) {
merged l=merge (todo, cdnaArray [k], todo_list);
}
}
if (!merged) {
push(Done-list, todo);
}
}
}
}

25. The method of claim 16, wherein said extending step comprises the step of:
performing a pairwise comparison of all remaining alignments, wherein for each pair, left-most and right-most overlapping exons are considered, and wherein if there are no conflicts on any of their hard edges, then short ended alignments are extended in such a way that they match longer alignments.

26. The method of claim 25, wherein if an alignment can be extended, then a new alignment is created, and it is further compared against all overlapping alignments, and wherein if an alignment cannot be extended, it gets placed on a Done list.

27. The method of claim 26, wherein after all comparisons are complete, said Done list is again purged of redundant alignments.

28. The method of claim 16, wherein said RNA derived sequence fragments comprise any of EST's, partial cDNA's and full-length cDNA's.

29. An apparatus for extending sparse alignment coverage for any combination of RNA derived sequence fragments, comprising:
a computer implemented algorithm for combining and catenating all combinations of overlapping alignments that agree with each other; and
a computer implemented algorithm for extending boundaries of overlapping alignments that agree with their first and last exons.

30. The apparatus of claim 29, further comprising:
computer implemented means for preprocessing and filtering.

31. The apparatus of claim 30, wherein said preprocessing and filtering means comprises means for:
reading a file containing alignments of exon annotations to genomic sequence; and
populating an array of data structures, one for each alignment, wherein each alignment is stored as a set of alignment blocks, each block representing a matching region between a given RNA and the genomic.

32. The apparatus of claim 31, wherein said blocks are considered exons, and gaps between them are considered introns.

33. The apparatus of claim 32, wherein 5′/3′ splice sites are referred to as hard edges, and the two ends of an alignment are referred to as soft ends.

34. The apparatus of claim 33, said preprocessing and filtering means further comprising means for:
determining if gaps are introns or inserts;
eliminating single exon alignments;
trimming soft ends of alignments; and
filtering out highly similar alignments.

35. The apparatus of claim 34, wherein gaps in alignment blocks that are less than or equal to twenty nucleotides are considered inserts instead of introns, and wherein two adjacent blocks are subsequently combined into one.

36. The apparatus of claim 35, wherein once an entire alignment has been read and all inserts removed, an alignment is discarded if it consists of only a single exon.

37. The apparatus of claim 36, wherein soft ends of an alignment are trimmed by ten nucleotides.

38. The apparatus of claim 37, wherein once all multi-exon alignments have been read and trimmed, there is a filtering step to cut down on running time.

39. The apparatus of claim 29, further comprising:
means for discarding similar alignments, wherein two alignments are similar if they are on a same strand and have similar number of agreeing exons.

40. The apparatus of claim 29, further comprising:
means for eliminating shorter alignments.

41. The apparatus of claim 33, further comprising:
means for removing similar alignments only if their soft ends are not on opposing sides of some other alignment's hard edge.

42. The apparatus of claim 39, wherein whenever a similar alignment is eliminated, an alignment that remains is stretched to widest possible soft end points of said two alignments to keep said alignments as long as possible, wherein for a set of many similar alignments whose soft ends are not near any hard edges, only one alignment remains after said filtering step; and wherein soft ends of a remaining alignment are widest possible among all of similar alignments.

43. The apparatus of claim 29, wherein said RNA derived sequence fragments comprise any of EST's, partial cDNA's and full-length cDNA's.

44. An apparatus for extending sparse alignment coverage for any combination of RNA derived sequence fragments, comprising:
a computer implemented algorithm for merging all combinations of overlapping alignments that agree with each other; and
a computer implemented algorithm for extending boundaries of overlapping alignments that agree with their first and last exons.

45. The apparatus of claim 44, wherein said merge algorithm performs a pairwise comparison of each overlapping RNA sequence on a same strand; wherein if their splice sites agree, then either a new, longer alignment is created out of said two sequences, or one of said alignments is labeled as redundant to the other and it is scheduled for deletion.

46. The apparatus of claim 45, wherein an alignment X is redundant if it agrees with another alignment Y, and soft ends of X fall completely within soft ends of Y, inclusive.

47. The apparatus of claim 46, wherein redundant alignments are not deleted until after said pairwise comparisons are complete.

48. The apparatus of claim 47, wherein a smaller alignment can be redundant with respect to another larger alignment and still be able to merge with other alignments that a larger alignment cannot; and wherein redundant pieces are still available for said pairwise comparisons, even though they are redundant with other alignments.

49. The apparatus of claim 48, wherein each newly created, merged alignment is also checked against other, unmerged alignments in a same way, either merging once again, becoming labeled as redundant, or causing other alignments to be labeled redundant.

50. The apparatus of claim 49, wherein whenever an alignment, merged or not, has been compared to all other alignments, and is neither redundant nor merged it is placed on a list of Done alignments.

51. The apparatus of claim 50, wherein once all comparisons have been finished, said Done alignments are again compared pairwise to check for redundancies.

52. The apparatus of claim 44, wherein said merging algorithm comprises the following:

| / | * cdnaAray = array of cDNA alignments stored by increasing | | |
|---|---|---|---|
| | * | start points | |
| | * todo list = a list of merged alignments that are | | |
| | * | scheduled to be compared to the other | * |

alignments in cdnaArray. Associated with each

| | |
|---|---|
| * | merged alignment is an integer |
| * | array index indicating where in cdnaArray the |
| * | comparisons should begin. |

*/
bool merged;
for l = 0 to sizeOf (cdnaArray) {
for j = (l + 1) to sizeOf (cdnaArray) {
merged = 0;
if ( overlap (cdnaArray[l], cdnaArray[j])) {
merged l = merge (cdnaArray[l], cdnaArray[j], todo_list);
```
  }
}
if (!merged) {
  push(Done_list cdnaArray[l]));
}
else {/* there must be a mered alignment on the todo_{—list */}
  while (todo_list !=NULL) {
```
todo = pop(todo_list);
merged = 0;
for k = todo,dtart to size Of (cdnaArray) {
if (overlap (todo, cdnaArray [k])) {
merged l=merge (todo, cdnaArray [k], todo_list*/
}
}
if (!merged) {
push(Done-list, todo);
}
}
}
}

53. The apparatus of claim 44, wherein said extending step comprises the step of:
performing a pairwise comparison of all remaining alignments, wherein for each pair, left-most and right-most overlapping exons are considered, and wherein if there are no conflicts on any of their hard edges, then short ended alignments are extended in such a way that they match longer alignments.

54. The apparatus of claim 53, wherein if an alignment can be extended, then a new alignment is created, and it is further compared against all overlapping alignments, and wherein if an alignment cannot be extended, it gets placed on a Done list.

55. The apparatus of claim 54, wherein after all comparisons are complete, said Done list is again purged of redundant alignments.

56. The apparatus of claim 44, wherein said RNA derived sequence fragments comprise any of EST's, partial cDNA's and full-length cDNA's.
